SEQUENCE LISTING (2 sequences)

SEQ ID NO: 1 - DNA, 2100 bp, Homo sp.
  source 1..2100: mol_type = genomic DNA
atgagccccgggctgctgctgctcggcagcgccgtcctgctcgccttcggcctctgctgcaccttcgtgcaccgcgctcgcagccgctacgagcacatccccgggccgccgcggcccagtttccttctaggacacctcccctgcttttggaaaaaggatgaggttggtggccgtgtgctccaagatgtgtttttggattgggctaagaagtatggacctgttgtgcgggtcaacgtcttccacaaaacctcagtcatcgtcacgagtcctgagtcggttaagaagttcctgatgtcaaccaagtacaacaaggactccaagatgtaccgtgcgctccagactgtgtttggtgagagactcttcggccaaggcttggtgtccgaatgcaactatgagcgctggcacaagcagcggagagtcatagacctggccttcagccggagctccttggttagcttaatggaaacattcaacgagaaggctgagcagctggtggagattctagaagccaaggcagatgggcagaccccagtgtccatgcaggacatgctgacctacaccgccatggacatcctggccaaggcagcttttgggatggagaccagtatgctgctgggtgcccagaagcctctgtcccaggcagtgaaacttatgttggagggaatcactgcgtcccgcaacactctggcaaagttcctgccagggaagaggaagcagctccgggaggtccgggagagcattcgcttcctgcgccaggtgggcagggactgggtccagcgccgccgggaagccctgaagaggggcgaggaggttcctgccgacatcctcacacagattctgaaagctgaagagggagcccaggacgacgagggtctgctggacaacttcgtcaccttcttcattgctggtcacgagacctctgccaaccacttggcgttcacagtgatggagctgtctcgccagccagagatcgtggcaaggctgcaggccgaggtggatgaggtcattggttctaagaggtacctggatttcgaggacctggggagactgcagtacctgtcccaggtcctcaaagagtcgctgaggctgtacccaccagcatggggcacctttcgcctgctggaagaggagaccttgattgatggggtcagagtccccggcaacaccccgctcttgttcagcacctatgtcatggggcggatggacacatactttgaggacccgctgactttcaaccccgatcgcttcggccctggagcacccaagccacggttcacctacttccccttctccctgggccaccgctcctgcatcgggcagcagtttgctcagatggaggtgaaggtggtcatggcaaagctgctgcagaggctggagttccggctggtgcccgggcagcgcttcgggctgcaggagcaggccacactcaagccactggaccccgtgctgtgcaccctgcggccccgcggctggcagcccgcacccccaccacccccctgctgagggggcctccaggcaggacgagactcctcgggcaagggccgtgcccgcccacctctgctgcccacggccacccacccttctcccctgccccgtcccctgggccacccttcacgctggcttccagcgggccctctgccgaccgcctgcttcacacccctcagcgctccctgtcgcctgcggactccatggcccttcctggactggcccttgcccaactcccagccaccaccactgtccctaccactgagcccttgcacaggccacttgctcagacgagacaccctaactcttgctcactccctaaagccctcttcaggggtcacctcctccaagaagccctccttgccaccccccgccggcaggggcccctcctctgtgctccctcggtcacctgtgctacctctaacaccacactgaccacactgtatcgtgagtgtccgttgacgtgaccaattgccctgccaggctgtcagcgcctcaagggtagggtctgcgtgtgatttgtctctgagccccctgtgcccacccagggcccggcacagagtcgatgctcaataaatgtgtgttgactgcaaaaaaaaaaaaaaaaaaaaaaaa

SEQ ID NO: 2 - protein, 500 aa, Homo sapiens
MSPGLLLLGSAVLLAFGLCCTFVHRARSRYEHIPGPPRPSFLLGHLPCFWKKDEVGGRVLQDVFLDWAKKYGPVVRVNVFHKTSVIVTSPESVKKFLMSTKYNKDSKMYRALQTVFGERLFGQGLVSECNYERWHKQRRVIDLAFSRSSLVSLMETFNEKAEQLVEILEAKADGQTPVSMQDMLTYTAMDILAKAAFGMETSMLLGAQKPLSQAVKLMLEGITASRNTLAKFLPGKRKQLREVRESIRFLRQVGRDWVQRRREALKRGEEVPADILTQILKAEEGAQDDEGLLDNFVTFFIAGHETSANHLAFTVMELSRQPEIVARLQAEVDEVIGSKRYLDFEDLGRLQYLSQVLKESLRLYPPAWGTFRLLEEETLIDGVRVPGNTPLLFSTYVMGRMDTYFEDPLTFNPDRFGPGAPKPRFTYFPFSLGHRSCIGQQFAQMEVKVVMAKLLQRLEFRLVPGQRFGLQEQATLKPLDPVLCTLRPRGWQPAPPPPPC

(19) United States
(12) Patent Application Publication
(10) Pub. No.: US 2026/0335385 A1
(43) Pub. Date: Oct. 29, 2026

(54) METHODS AND PHARMACEUTICAL COMPOSITION FOR THE TREATMENT OF ALPHA-SYNUCLEINOPATHIES

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); CENTRE NATIONAL DE LA RECHERCHE SCIENTIFIQUE, Paris (FR); ICM (INSTITUT DU CERVEAU ET DE LA MOELLE EPINIÈRE), Paris (FR); SORBONNE UNIVERSITÉ, Paris (FR); THE UNIVERSITY OF OSAKA, Osaka (JP)

(72) Inventors: Nathalie CARTIER-LACAVE, Paris (FR); Corinne BESNARD-GUERIN, Paris (FR); Lisa ROUSSELOT, Paris (FR); Hideki MOCHIZUKI, Osaka (JP); Satoru TADA, Osaka (JP)

(21) Appl. No.: 19/119,214

(22) PCT Filed: Oct. 13, 2023
(86) PCT No.: PCT/EP2023/078486

§ 371 Date: Apr. 8, 2025

(30) Foreign Application Priority Data
Oct. 14, 2022 (EP) 22306556.6

(51) Int. Cl. A61K 35/761; A61K 35/763; A61K 38/44; A61P 25/16

(52) U.S. Cl. CPC A61K 35/761; A61K 35/763; A61K 38/44; A61P 25/16; C12Y 114/13098

(57) ABSTRACT

The present invention relates to the treatment alpha-synucleinopathies. In this study, the inventors showed that restoring brain cholesterol pathway and defective autophagy by AAV-CYP46A1 delivery, as evidenced in several neurodegenerative pathologies, could be a relevant therapeutic approach in alpha-synucleinopathies and particularly in PD. Thus the present invention relates to a vector for use in the treatment of alpha-synucleinopathies, which vector comprises the full sequence of cholesterol 24-hydroxylase encoding nucleic acid.

### FIELD OF THE INVENTION

The present invention relates to a vector for use in the treatment of alpha-synucleinopathies, which vector comprises the full sequence of cholesterol 24-hydroxylase encoding nucleic acid.

### BACKGROUND OF THE INVENTION

Disturbances in brain cholesterol metabolism play an important role in neurodegenerative diseases including Alzheimer Disease (AD), Huntington Disease (HD), Spinocerebellar Ataxias (Sca) and Parkinson Disease (PD). In particular, the cholesterol 24-hydroxylase (CYP46A1), the key neuronal enzyme that converts excess of cholesterol into 24-hydroxycholesterol and ensures the turnover of brain cholesterol is decreased in affected brain regions in patients with AD, HD, Sca3. These diseases have in common the accumulation of misfolded proteins that aggregate and are toxic for neuronal cells. Studies from NEUROGENCELL lab showed that restoring CYP46A1 and cerebral cholesterol metabolism is a potentially relevant therapeutic strategy for AD (Hudry E, et al. 2010 and Burlot et al. 2015), HD, SCA and ALS (Wurtz G; et al. 2022). A single injection of an adeno-associated vector (AAV) encoding for CYP46A1 in affected brain regions effectively reduces aggregated proteins, restores several key cellular functions such as transcription activity, vesicular transport, synaptic transmission, endocytosis and autophagy, and promotes neuronal survival. Interestingly, in PD, a decrease in plasmatic 24-hydroxycholesterol levels in patients, suggests a possible alteration in brain cholesterol metabolism. In addition, studies demonstrated that cholesterol and alpha-synuclein are closely linked in cellular compartments. Moreover, neuronal autophagy defects are believed to be crucially involved in PD pathogenesis.

### SUMMARY OF THE INVENTION

In this study, the inventors showed that restoring brain cholesterol pathway and defective autophagy by AAV-CYP46A1 delivery, as evidenced in several neurodegenerative pathologies, could be a relevant therapeutic approach in alpha-synucleinopathies and particularly in PD.

Thus, the invention relates to a vector for use in the treatment of alpha-synucleinopathies, which vector comprises the full sequence of cholesterol 24-hydroxylase encoding nucleic acid.

Particularly, the invention is defined by its claims.

### DETAILED DESCRIPTION OF THE INVENTION

### The CYP46A1 Sequences and Used Thereof

A first object of the invention relates to a vector for use in the treatment of alpha-synucleinopathies in a subject in need thereof, which vector comprises the full sequence of cholesterol 24-hydroxylase encoding nucleic acid.

In a particular embodiment, the invention relates to a vector for use in the treatment of Lewy body diseases in a subject in need thereof, which vector comprises the full sequence of cholesterol 24-hydroxylase encoding nucleic acid.

As used herein, the term “alpha-synucleinopathies” denotes incorporates clinical diseases such as Parkinson disease (PD), PD with dementia, dementia with Lewy bodies, and multiple-system atrophy (MSA).

In some embodiment, the alpha-synucleinopathy is not Alzheimer Disease (AD).

As used herein, the term “gene” refers to a polynucleotide containing at least one open reading frame that is capable of encoding a particular polypeptide or protein after being transcribed or translated.

As used herein, the terms “coding sequence” or “a sequence which encodes a particular protein” or “encoding nucleic acid”, denotes a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

The CYP46A1 gene encodes cholesterol 24-hydroxylase. This enzyme is a member of the cytochrome P450 superfamily. A cDNA sequence for CYP46A1 is disclosed in Genbank Access Number AF094480.1 (SEQ ID NO:1). The amino acid sequence is shown in SEQ ID NO: 2.

SEQ ID NO: 1:
atgagccccg ggctgctgct gctcggcagc gccgtcctgc tcgccttcgg cctctgctgc accttcgtgc
accgcgctcg cagccgctac gagcacatcc ccgggccgcc gcggcccagt ttccttctag gacacctccc ctgcttttgg
aaaaaggatg aggttggtgg ccgtgtgctc caagatgtgt ttttggattg ggctaagaag tatggacctg ttgtgcgggt
caacgtcttc cacaaaacct cagtcatgt cacgagtcct gagtcggtta agaagttcct gatgtcaacc aagtacaaca
aggactccaa gatgtaccgt gcgctccaga ctgtgtttgg tgagagactc ttcggccaag gcttggtgtc cgaatgcaac
tatgagcgct ggcacaagca gcggagagtc atagacctgg ccttcagccg gagctccttg gttagcttaa tggaaacatt
caacgagaag gctgagcagc tggtggagat tctagaagcc aaggcagatg ggcagacccc agtgtccatg caggacatgc
tgacctacac cgccatggac atcctggcca aggcagcttt tgggatggag accagtatgc tgctgggtgc ccagaagcct
ctgtcccagg cagtgaaact tatgttggag ggaatcactg cgtcccgcaa cactctggca aagttcctgc cagggaagag
gaagcagctc cgggaggtcc gggagagcat tcgcttcctg cgccaggtgg gcagggactg ggtccagcgc cgccgggaag
ccctgaagag gggcgaggag gttcctgccg acatcctcac acagattctg aaagctgaag agggagccca ggacgacgag
ggtctgctgg acaacttcgt caccttcttc attgctggtc acgagacctc tgccaaccac ttggcgttca cagtgatgga
gctgtctcgc cagccagaga tcgtggcaag gctgcaggcc gaggtggatg aggtcattgg ttctaagagg tacctggatt
tcgaggacct ggggagactg cagtacctgt cccaggtcct caaagagtcg ctgaggctgt acccaccagc atggggcacc
tttcgcctgc tggaagagga gaccttgatt gatggggtca gagtccccgg caacaccccg ctcttgttca gcacctatgt
catggggcgg atggacacat actttgagga cccgctgact ttcaaccccg atcgcttcgg ccctggagca cccaagccac
ggttcaccta cttccccttc tccctgggcc accgctcctg catcgggcag cagtttgctc agatggaggt gaaggtggtc
atggcaaagc tgctgcagag gctggagttc cggctggtgc ccgggcagcg cttcgggctg caggagcagg ccacactcaa
gccactggac cccgtgctgt gcaccctgcg gccccgcggc tggcagcccg cacccccacc acccccctgc tgagggggcc
tccaggcagg acgagactcc tcgggcaagg gccgtgcccg cccacctctg ctgcccacgg ccacccaccc ttctcccctg
ccccgtcccc tgggccaccc ttcacgctgg cttccagcgg gccctctgcc gaccgcctgc ttcacacccc tcagcgctcc
ctgtcgcctg cggactccat ggcccttcct ggactggccc ttgcccaact cccagccacc accactgtcc ctaccactga
gcccttgcac aggccacttg ctcagacgag acaccctaac tcttgctcac tccctaaagc cctcttcagg ggtcacctcc
tccaagaagc cctccttgcc accccccgcc ggcaggggcc cctcctctgt gctccctcgg tcacctgtgc tacctctaac
accacactga ccacactgta tcgtgagtgt ccgttgacgt gaccaattgc cctgccaggc tgtcagcgcc tcaagggtag
ggtctgcgtg tgatttgtct ctgagccccc tgtgcccacc cagggcccgg cacagagtcg atgctcaata aatgtgtgtt
gactgcaaaa aaaaaaaaaa aaaaaaaaaa
SEQ ID NO: 2:
MSPGLLLLGS AVLLAFGLCC TFVHRARSRY EHIPGPPRPS FLLGHLPCFW
KKDEVGGRVL QDVFLDWAKK YGPVVRVNVF HFTSVIVTSP ESVKKFLMST
KYNKDSKMYR ALQTVFGERL FGQGLVSECN YERWHKQRRV IDLAFSRSSL
VSLMETFNEK AEQLVEILEA KADGQTPVSM QDMLTYTAMD ILAKAAFGME
TSMLLGAQKP LSQAVKLMLE GITASRNTLA KFLPGKRKQL REVRESIRFL
RQVGRDWVQR RREALKRGEE VPADILTQIL KAEEGAQDDE GLLDNFVTFF
IAGHETSANH LAFTVMELSR QPEIVARLQA EVDEVIGSKR YLDFEDLGRL
QYLSQVLKES LRLYPPAWGT FRLLEEETLI DGVRVPGNTP LLFSTYVMGR
MDTYFEDPLT FNPDRFGPGA PKPRFTYFPF SLGHRSCIGQ QFAQMEVKVV
MAKLLQRLEF RLVPGQRFGL QEQATLKPLD PVLCTLRPRG WQPAPPPPPC

In a preferred embodiment, the invention provides a nucleic acid construct comprising sequence SEQ ID N°1 or a variant thereof for the treatment of alpha-synucleinopathies.

The variants include, for instance, naturally-occurring variants due to allelic variations between individuals (e.g., polymorphisms), alternative splicing forms, etc. The term variant also includes CYP46A1 gene sequences from other sources or organisms. Variants are preferably substantially homologous to SEQ ID No 1, i.e., exhibit a nucleotide sequence identity of typically at least about 75%, preferably at least about 85%, more preferably at least about 90%, more preferably at least about 95% with SEQ ID No 1. Variants of a CYP46A1 gene also include nucleic acid sequences, which hybridize to a sequence as defined above (or a complementary strand thereof) under stringent hybridization conditions. Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

As used herein, the term “subject” denotes a mammal, such as a rodent, a feline, a canine, and a primate. Particularly, a subject according to the invention is a human. Particularly, a subject according to the invention is a human with an alpha-synucleinopathie.

As used herein, the term “treatment” or “treat” refers to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness (e.g., the pattern of dosing used during therapy). A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The invention also relates to a method of treating alpha-synucleinopathies in a subject in need thereof, said method comprising administering to the subject a therapeutically effective amount of a vector which comprises the full sequence of cholesterol 24-hydroxylase encoding nucleic acid.

Non Viral Vectors In a preferred embodiment, the vector use according to the invention is a non viral vector. Typically, the non viral vector may be a plasmid encoding CYP46A1.

### Viral Vectors

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction.

The terms “Gene transfer” or “gene delivery” refer to methods or systems for reliably inserting foreign DNA into host cells. Such methods can result in transient expression of non integrated transferred DNA, extrachromosomal replication and expression of transferred replicons (e. g., episomes), or integration of transferred genetic material into the genomic DNA of host cells.

Examples of viral vector include adenoviral, retroviral, lentiviral, herpesvirus and adeno-associated virus (AAV) vectors.

Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. Nos. 5,882,877, 6,013,516, 4,861,719, U.S. Pat. No. 5,278,056 and WO94/19478.

In a preferred embodiment, adeno-associated viral (AAV) vectors are employed.

In another preferred embodiment, the AAV vector is AAV1, AAV2, AAV3, AAV4, AAV5, AAV6, AAV7, AAV8, AAV9, AAVrh10 and all variants of AAV9, including AAVPHP.eB, AAV3B, AAV-2i8, Rh74, AAV capB10 or any other serotypes of AAV that can infect human, monkeys or other species.

In a more preferred embodiment, the AAV vector is an AAVrh10.

By an “AAV vector” is meant a vector derived from an adeno-associated virus serotype, including without limitation, AAV-1, AAV-2, AAV-3, AAV-4, AAV-5, AAV6, etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e. g., functional ITRs) of the virus. The ITRs need not be the wild-type nucleotide sequences, and may be altered, e. g, by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the DNA of interest (i.e. the CYP46A1 gene) and a transcriptional termination region.

The control elements are selected to be functional in a mammalian cell. The resulting construct which contains the operatively linked components is bounded (5′ and Y) with functional AAV ITR sequences. By “adeno-associated virus inverted terminal repeats” or “AAVITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e. g., Kotin, 1994; Berns, KI “Parvoviridae and their Replication” in Fundamental Virology, 2nd Edition, (B. N. Fields and D. M. Knipe, eds.) for the AAV-2 sequence. As used herein, an “AAV ITR” does not necessarily comprise the wild-type nucleotide sequence, but may be altered, e. g., by the insertion, deletion or substitution of nucleotides. Additionally, the AAV ITR may be derived from any of several AAV serotypes, including without limitation, AAV-1, AAV-2, AAV-3, AAV-4, AAV-5, AAV6, etc. Furthermore, 5′ and 3′ITRs which flank a selected nucleotide sequence in an AAV vector need not necessarily be identical or derived from the same AAV serotype or isolate, so long as they function as intended, i. e., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV-1, AAV-2, AAV-3, AAV-4, AAV 5, AAV6, etc. Furthermore, 5′ and 3′ITRs which flank a selected nucleotide sequence in an AAV expression vector need not necessarily be identical or derived from the same AAV serotype or isolate, so long as they function as intended, i. e., to allow for excision and rescue of the sequence of interest from a host cell genome or vector, and to allow integration of the DNA molecule into the recipient cell genome when AAV Rep gene products are present in the cell.

Particularly preferred are vectors derived from AAV serotypes having tropism for and high transduction efficiencies in cells of the mammalian CNS, particularly neurons. A review and comparison of transduction efficiencies of different serotypes is provided in Cearley C N et al., 2009. In one preferred example, AAV2 based vectors have been shown to direct long-term expression of transgenes in CNS, preferably transducing neurons. In other nonlimiting examples, preferred vectors include vectors derived from AAVrh10 serotype, which have also been shown to transduce cells of the CNS and particularly the striatum or the substantia nigra (SN) and more particularly the substantia nigra pars compacta (SNpc).

The selected nucleotide sequence is operably linked to control elements that direct the transcription or expression thereof in the subject in vivo. Such control elements can comprise control sequences normally associated with the selected gene.

Alternatively, heterologous control sequences can be employed. Useful heterologous control sequences generally include those derived from sequences encoding mammalian or viral genes. Examples include, but are not limited to, the phophoglycerate kinase (PKG) promoter, CAG, neuronal promoters, promoter of Dopamine-1 receptor and Dopamine-2 receptor, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e. g., Stratagene (San Diego, CA). For purposes of the present invention, both heterologous promoters and other control elements, such as CNS-specific and inducible promoters, enhancers and the like, will be of particular use.

Examples of heterologous promoters include the CMV promoter. Examples of CNS specific promoters include those isolated from the genes from myelin basic protein (MBP), glial fibrillary acid protein (GFAP), and neuron specific enolase (NSE).

Examples of inducible promoters include DNA responsive elements for ecdysone, tetracycline, hypoxia andaufin.

The AAV expression vector which harbors the DNA molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e. g., U.S. Pat. Nos. 5,173,414 and 5,139,941; International Publications Nos. WO 92/01070 (published 23 Jan. 1992) and WO 93/03769 (published 4 Mar. 1993); Lebkowski et al., 1988; Vincent et al., 1990; Carter, 1992; Muzyczka, 1992; Kotin, 1994; Shelling and Smith, 1994; and Zhou et al., 1994. Alternatively, AAV ITRs can be excised from the viral genome or from an AAV vector containing the same and fused 5′ and 3′ of a selected nucleic acid construct that is present in another vector using standard ligation techniques. AAV vectors which contain ITRs have been described in, e. g., U.S. Pat. No. 5,139,941. In particular, several AAV vectors are described therein which are available from the American Type Culture Collection (“ATCC”) under Accession Numbers 53222,53223, 53224,53225 and 53226. Additionally, chimeric genes can be produced synthetically to include AAV ITR sequences arranged 5′ and 3′ of one or more selected nucleic acid sequences. Preferred codons for expression of the chimeric gene sequence in mammalian CNS cells can be used. The complete chimeric sequence is assembled from overlapping oligonucleotides prepared by standard methods. See, e. g., Edge, 1981; Nambair et al., 1984; Jay et al., 1984. In order to produce rAAV virions, an AAV expression vector is introduced into a suitable host cell using known techniques, such as by transfection. A number of transfection techniques are generally known in the art. See, e. g., Graham et al., 1973, Sambrook et al. (1989) Molecular Cloning, a laboratory manual, Cold Spring Harbor Laboratories, New York, Davis et al. (1986) Basic Methods in Molecular Biology, Elsevier, and Chu et al., 1981. Particularly suitable transfection methods include calcium phosphate co-precipitation (Graham et al., 1973), direct microinjection into cultured cells (Capecchi, 1980), electroporation (Shigekawa et al., 1988), liposome mediated gene transfer (Mannino et al., 1988), lipid-mediated transduction (Felgner et al., 1987), and nucleic acid delivery using high-velocity microprojectiles (Klein et al., 1987).

For instance, a preferred viral vector, such as the AAVrh10, comprises, in addition to a cholesterol 24-hydroxylase encoding nucleic acid sequence, the backbone of AAV vector with ITR derived from AAV-2, the promoter, such as the mouse PGK (phosphoglycerate kinase) gene or the cytomegalovirus/β-actin hybrid promoter (CAG) consisting of the enhancer from the cytomegalovirus immediate gene, the promoter, splice donor and intron from the chicken β-actin gene, the splice acceptor from rabbit β-globin, or any neuronal promoter such as the promoter of Dopamine-1 receptor or Dopamine-2 receptor with or without the wild-type or mutant form of woodchuck hepatitis virus post-transcriptional regulatory element (WPRE).

### Delivery of the Vectors

It is herein provided a method for treating alpha-synucleinopathies in a subject, said method comprising:
(a) providing a vector as defined above, which comprises a cholesterol 24-hydroxylase encoding nucleic acid; and
(b) delivering the vector to the central nervous system (CNS), either directly to the substantia nigra or indirectly through retrograde transport from the putamen of the subject, whereby said vector transduces cells in the CNS, and whereby cholesterol 24-hydroxylase is expressed by the transduced cells at a therapeutically effective level.

In a particular embodiment, the delivery of the vector in realized in the striatum or in the SNpc.

Methods of delivery of vectors to neurons and/or astrocytes includes generally any method suitable for delivery vectors to the neurons and/or astrocytes such that at least a portion of cells of a selected synaptically connected cell population is transduced. The vector may be delivered to any cells of the central nervous system, or both. Generally, the vector is delivered to the cells of the central nervous system, including for example cells of the spinal cord, brainstem (medulla, pons, and midbrain), cerebellum, diencephalon (thalamus, hypothalamus), telencephalon (corpus striatum, substantia nigra, cerebral cortex, or, within the cortex, the occipital, temporal, parietal or frontal lobes), or combinations thereof, or preferably any suitable subpopulation thereof. Further preferred sites for delivery include the ruber nucleus, corpus amygdaloideum, entorhinal cortex and neurons in ventralis lateralis, or to the anterior nuclei of the thalamus.

To deliver the vector specifically to a particular region and to a particular population of cells of the CNS, the vector may be administered by intracerebral injection. For example, patients have the stereotactic frame base fixed in place (screwed into the skull). The brain with stereotactic frame base (MRI compatible with fiducial markings) is imaged using high resolution MRI. The MRI images are then transferred to a computer which runs stereotactic software. A series of coronal, sagittal and axial images are used to determine the target (site of AAV vector injection) and trajectory. The software directly translates the trajectory into 3 dimensional coordinates appropriate for the stereotactic frame. Alternatively, neuronavigation can be used instead of the stereotactic procedure, depending on neurosurgeon preference. Burr holes are drilled above the entry site and the stereotactic apparatus positioned with the needle implanted at the given depth. The AAV vector is then injected at the target sites. Since the AAV vector integrates into the target cells, rather than producing viral particles, the subsequent spread of the vector is minor, and mainly a function of passive diffusion from the site of injection and of course the desired transsynaptic transport, prior to integration. The degree of diffusion may be controlled by adjusting the ratio of vector to fluid carrier.

Additional routes of administration may also comprise local application of the vector under direct visualization, e. g., superficial cortical application, or other nonstereotactic application. The vector may be delivered intraparenchymally, intracisternally, intrathecally or intravenously and een intravenously couple with focused ultrasound to improve blood brain barrier crossing.

The target cells of the vectors of the present invention are cells of the central nervous systems of a subject afflicted with alpha-synucleinopathies and more particularly the cells of the SNpc. However, neurons from the striatum are also targeted cells as they project into the SNpc and allow indirect tard-getting of the cells with less invasive surgery. Preferably the subject is a human being, generally an adult.

However the invention encompasses delivering the vector to biological models of the disease. In that case, the biological model may be any mammal at any stage of development at the time of delivery, e. g., embryonic, fetal, infantile, juvenile or adult, preferably it is an adult. Furthermore, the target CNS cells may be essentially from any source, especially nonhuman primates and mammals of the orders Rodenta (mice, rats, rabbit, hamsters), Carnivora (cats, dogs), and Arteriodactyla (cows, pigs, sheep, goats, horses) as well as any other non-human system (e. g. zebrafish model system).

Preferably, the method of the invention comprises intracerebral administration through stereotaxic injections. However, other known delivery methods may also be adapted in accordance with the invention. For example, for a more widespread distribution of the vector across the CNS, it may be injected into the cerebrospinal fluid, e. g., by lumbar puncture. To direct the vector to the peripheral nervous system, it may be injected into the spinal cord or into the peripheral ganglia, or the flesh (subcutaneously or intramuscularly) of the body part of interest. In certain situations the vector can be administered via an intravascular approach. For example, the vector can be administered intra-arterially (carotid) in situations where the blood-brain barrier is disturbed or not disturbed. Moreover, for more global delivery, the vector can be administered during the “opening” of the blood-brain barrier achieved by infusion of hypertonic solutions including mannitol.

The vectors used herein may be formulated in any suitable vehicle for delivery. For instance they may be placed into a pharmaceutically acceptable suspension, solution or emulsion. Suitable mediums include saline and liposomal preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes.

The preferred doses and regimen may be determined by a physician, and depend on the age, sex, weight, of the subject, and the stage of the disease. As an example, for delivery of cholesterol 24-hydroxylase using a viral expression vector, each unit dosage of cholesterol 24-hydroxylase expressing vector may comprise 2.5 to 500 μl of a composition including a viral expression vector in a pharmaceutically acceptable fluid at a concentration ranging from 10¹¹ to 10¹⁶ viral genome per ml for example.

### Pharmaceutical Composition

A second object of the invention concerns a pharmaceutical composition for use in the treatment of alpha-synucleinopathies in a subject in need thereof which comprises a therapeutically effective amount of a vector according to the invention.

Particularly, the invention concerns a pharmaceutical composition for use in the treatment of Lewy body diseases in a subject in need thereof, which comprises a therapeutically effective amount of a vector according to the invention.

By a “therapeutically effective amount” is meant a sufficient amount of the vector of the invention to treat alpha-synucleinopathies at a reasonable benefit/risk ratio applicable to any medical treatment.

It will be understood that the total daily dosage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range per adult per day. The therapeutically effective amount of the vector according to the invention that should be administered, as well as the dosage for the treatment of a pathological condition with the number of viral or non-viral particles and/or pharmaceutical compositions of the invention, will depend on numerous factors, including the age and condition of the patient, the severity of the disturbance or disorder, the method and frequency of administration and the particular peptide to be used.

The presentation of the pharmaceutical compositions that contain the vector according to the invention may be in any form that is suitable for intraparenchymal, intracisternal, intracerebral, intrathecal, intraventricular or intravenous administration.

In the pharmaceutical compositions of the present invention for intramuscular, intravenous, intracerebral, intrathecal, intraventicular, intraparenchymal or intracisternal administration, the active principle, alone or in combination with another active principle, can be administered in a unit administration form, as a mixture with conventional pharmaceutical supports, to animals and human beings.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The vector according to the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

Multiple doses can also be administered.

The invention will be further illustrated by the following figures and examples. However, these examples and figures should not be interpreted in any way as limiting the scope of the present invention.

### FIGURES

FIG. 1: CYP46A1 overexpression decreases the levels of alpha-synuclein protein in the human SH-SY5Y cell line by promoting the activation of autophagy.

A. Representative western blot from SH-SY5Ycells co-transfected with a plasmid expressing WT alpha-synuclein and a plasmid encoding either CYP46A1 or GFP. Co-transfected cells were treated with specific autophagy inhibitors 3-methyladenine (3MA) and bafilomycin A1 (Baf).

B. Densitometric analysis showed that CYP46A1 expression leads to a significant decrease in alpha-synuclein levels even in presence of autophagy inhibitors.

C. Densitometric analysis showed a significant increase in LC3B-II levels compared to LC3B-I levels suggesting an autophagy activation upon CYP46A1 expression.

FIG. 2: Design of the POC in the model with alpha-synuclein fibrils injection.

FIG. 3: Endogenous CYP46A1 protein level in MPTP mouse model.

FIG. 4: Unbiased stereological estimation of TH-positive neurons count in the mouse SNpc 30 days after the last MPTP injection.

FIG. 5: TH+ neurons count by level in the mouse SNpc 30 days after the last MPTP injection.

FIG. 6: Protection of dopaminergic neurons loss with CYP46A1 in the C3 SNpc level 30 days after the last MPTP injection.

FIG. 7: Alpha-synuclein pathology is significantly reduced when mice are treated with an AAV-CYP46A1 vector.
A. Bright-field microscopy images showing mouse midbrains with phosphorylated alpha-synuclein immunolabelled
B. Quantification of phospho-alpha-synuclein positive cells in the midbrain of alpha-synuclein A53T mice treated or not with an AAV-CYP46A1

FIG. 8: Nigrostriatal degeneration is significantly reduced by AAV-CYP46A1 in the Neuromelanin PD mouse model
A. Unbiased stereological estimation of TH-positive neurons in the mouse SNpc 2 months after AAVs injection
B. Quantification of dopaminergic terminals in the mouse striatum by optical density measurement

FIG. 9: The motor function of Neuromelanin mice treated with AAV-CYP46A1 is significantly less impaired because of the preservation of nigrostriatal degeneration.
A. Mean latency to fall at the Rotarod test during the months following AAVs injection
B. Graph showing forelimb stride length assessed at the Catwalk test at 2 months post-injection of AAVs
C. Graph showing hind limbs stride length assessed at the Catwalk test at 2 months post-injection of AAVs

### EXAMPLES

### Example 1: In Vitro Model of Human Alpha-Synuclein Overexpression

### Material and Methods

### Alpha-Synuclein Untagged Constructs

Alpha-synuclein (WT or A53T) human cDNA were amplified by PCR from previously described vectors kindly provided by Addgene (plasmids #102361 and 105727). The PCR products were cloned in the HindIII/Xba I sites of the pcDNA3.1 vector.

### Cell Culture and Transfection

Human neuroblastoma (SH-SY5Y) cells were grown in DMEM with Glutamax (Gibco) with 10% fetal bovine serum (Gibco), 100 U/ml penicillin, and 100 μg/ml streptomycin (Gibco) in 5% CO2 at 37° C. Cells were transiently transfected with plasmids using Lipofectamine 2000 (Invitrogen), according to the manufacturer's instructions.

pcDNA3-EGFP and pAAV-HA-CYP46A1 plasmids were provided by Addgene (plasmid #13031). Experiments were initiated 24 h after transfection. For autophagy inhibition, cells were incubated for 24H by addition of 3 methyladenine or bafilomycin A1. 3 methyladenine (Millipore) or Bafilomycin A1 (Sigma) were dissolved respectively in cell medium to a final concentration of 5 mM or 50 nM

### Protein Extraction and Western-Blot Analysis

Cell pellets were homogenized in TENGEN buffer (150 mM NaCl, 50 mM Tris pH7.4, 1% Triton X-100) containing fresh protease inhibitor for 30 min at 4° C. Whole lysates were clarified by centrifugation at 10,000 rpm for 15 min. Proteins were quantified using Pierce BCA Protein Assay Kit (Thermo Scientific) by comparison with bovine serum albumin (BSA) standards. Equal volumes of each sample were separated by SDS-PAGE. Protein samples were denatured for 3 min at 95° C. and separated on 4-20% SDS-polyacrylamide gels and transferred to nitrocellulose membranes (Bio-Rad)). Membranes were blocked with 5% BSA in Tris-buffered saline (TBS) pH 8 for 1 h, followed by incubation with primary antibodies (see below) for 16 h at 4° C. (1:1000). Membranes were washed for 10 min three times in TBS with 0.1% Tween-20, incubated with LI-COR fluorescent secondary antibodies (Dylight 680 and Dylight 800) for 1 h at room temperature (1:7500), and washed three more times with TBS with 0.1% Tween-20. A Li-COR Odyssey fluorescence scanner was used to capture images of the membranes.

### Antibodies

The following primary antibodies were used: rabbit anti-actin (Sigma, A2066); rabbit anti-GFP (Sigma, SAB301138); rabbit anti-HA (Cell Signaling, 3724) or mouse anti-alpha-synuclein antibody (Invitrogen, AHB0261). Secondary antibodies for immunoblotting were the appropriate goat anti-mouse or anti-rabbit IRDye 680RD or 800CW IgG (Odyssey LI-COR)

### Results:

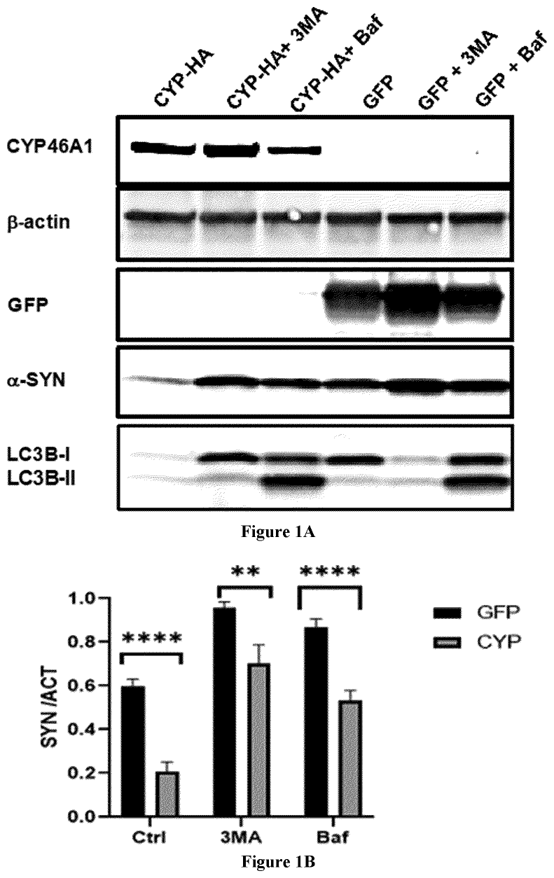

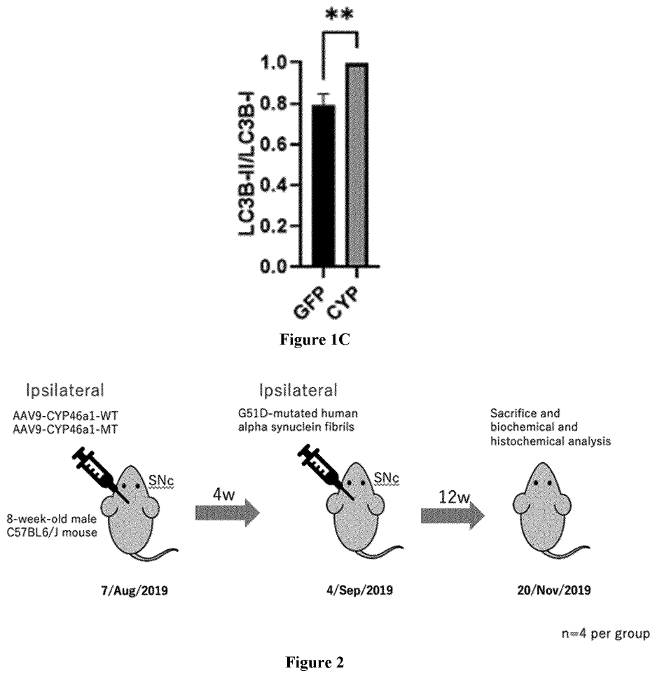

The inventors investigated in vitro the impact of CYP46A1 overexpression on wild-type (WT) α-syn. To this end, neuroblastoma (SH-SY5Y) cells were co-transfected with a plasmid expressing WT α-syn and a plasmid encoding either CYP46A1 or GFP. Western blot analysis showed a statistically significant reduction in WT α-syn protein levels with CYP46A1 compared to the GFP (FIGS. 1A, 1B). CYP46A1 overexpression is also able to decrease the level of mutated A53T α-syn proteins in SH-SY5Y cells after co-transfection with a A53T a-syn plasmid (data not shown). To further investigate the mechanisms of CYP46A1-mediated effects on α-syn clearance, co-transfected SH-SY5Y cells were treated with specific autophagy inhibitors 3-methyladenine (3MA) and bafilomycin A1 (Baf). Treatment of cells with 3-MA or Baf resulted in a clear increase of WT a-syn in cells even in the presence of CYP46A1 (FIGS. 1A, 1B). Overexpression of CYP46A1 significantly increased levels of LC3B-II compared to LC3B-I, suggesting that CYP46A1 promotes autophagic degradation of α-syn (FIGS. 1A, 1C).

This suggests that CYP46A1, that was previously shown to restore impaired autophagy pathway by improving the maturation of autophagosomes, could have a beneficial effect in PD mice models, particularly in the synucleinopathy models.

Next, the inventors validated this hypothesis in vivo in PD mouse models of PD.

### Example 2: Alpha-Synuclein Fibrils Model

### Material and Methods

In collaboration with Satoru Tada (Japan), the inventors generated a proof of concept in mice that received injection of mutated alpha-synuclein fibrils. Eight weeks old wild type mice received an unilateral injection of AAV9 encoding either a WT hCYP46A1 or a mutated hCYP46A1 which is not functionally active as a control in the substantia nigra par compacta (SNc). At eight weeks, animals received unilateral injection of G51D mutated human alphasynculein fibrils in the SNc. Mice were sacrificed at 12 weeks for analysis (FIG. 2).

### Results:

In mice that received the hCYP46A1 WT, a significant prevention of TH positive cells was demonstrated, with a total number of TH positive cells quite similar to normal levels (data not shown).

### Example 3: Subchronic MPTP Poisoning Model

### Material and Methods

### Subchronic MPTP Intoxication

MPTP administration is performed at least 16 days after the stereotaxic delivery of AAV-CYP46A1 vector so that animals have the necessary recovery time and the vector is well expressed. Animals poisoned with this neurotoxin are transferred, the day before the poisoning protocol, to the chemical area of the animal facility and placed on a ventilated rack. The animals are weighed and identified with an indelible marker. They are placed in disposable plastic cages (MPTP constraints) with filter cover. A piece of cotton wool intended for nesting is placed in the cage in order to improve the environment of the animals. On the day of the intoxication, the MPTP is diluted in physiological serum (NaCl 0.9%) at a concentration of 2.5 mg/ml. The volume injected does not exceed 280 μl/injection/mouse. The intraperitoneal injections of MPTP or physiological serum are carried out using 1 ml insulin syringes fitted with a 12.7×0.33 needle. Five injections of MPTP (30 mg/kg) at the rate of one injection per day are given over 5 consecutive days. The injections are alternated on the left and right side of the animal. The cages are placed in another ventilated cabinet, the temperature of which is set at 28° C. In fact, it was found that mice poisoned with MPTP according to the diet indicated in this procedure suffered from hypothermia and heart failure for a limited period of time (less than 12 hours). These peripheral side effects can lead to the death of 1 to 10% of the workforce within 12 hours after poisoning, depending on the experience. The 10% rate is not fixed and corresponds to a high limit range. Furthermore, this experimental procedure does not cause severe behavior (such as convulsions), significant brain damage, or ischemia under narcosis. The hypothermia observed in the intoxicated animals justifies that they be placed at a higher temperature (28° C.) and under observation throughout the procedure and 72 hours beyond the poisoning, without changing the litter. Once the 72 hours have elapsed, the animals will be transferred to clean cages and returned to a chemical housing area, but at standard animal room temperature and under observation.

### AAV Plasmid Design and Vector Production

AAV vectors were produced and purified by Atlantic Gene therapies (INSERM U1089, Nantes, France). Vector production has been described elsewhere (Hudry et al., 2010). The viral constructs for AAVrh. 10-GFP and AAVrh.10-CYP46A1 contained the expression cassette consisting of the human Cyp46algenes, driven by a CMV early enhancer/chicken β-actin (CAG) synthetic promoter (CAG) surrounded by inverted terminal repeats (ITR) sequences of AAV2.

### Tissue Collection

Mice were anesthetized with pentobarbital (Euthasol 180 mg/kg) solution and perfused transcardially with phosphate buffered saline (PBS). Brains were collected and post-fixed in PFA 4% prior to inclusion for histology or immediately frozen in liquid nitrogen for biomolecular analysis. Different CNS tissues (ventral midbrain, striatum, frontal cortex and cerebellum) were dissected and crushed using a FastPrep-24™ homogenizer (MP Biomedicals) to extract proteins or RNA for western-blotting and RT-qPCR analys

### Histological Analysis

Coronal brain sections (20 μm) were cut on a freezing microtome (Leica), collected serially in a cryoprotective solution, and stored at −20° C. until use. Immunostainings were carried out on frozen free-floating sections. Slices were washed in PBS 0,1M and blocked in PBS 0.1M Triton 0.03% containing 5% normal goat serum (NGS, Gibco) for 1 hour. Sections were then incubated with the respective primary antibody (see Antibodies section) in PBS-Triton 0.03% containing 3% NGS over night at 4° C. After three successive washes of 5 minutes in PBS, brain slices wete incubated for 1 hour and 30 minutes with seconday Alexa Fluor-conjugated (Invitrogen) antibodies directed against the species of the primary antibody at room temperature. Slices were stained in a DAPI solution, mounted in Fluoromount-G™ (Invitrogen) and conserved at 4° C. until microscopy analysis. The immunoassayed slides were then scanned using a slide scanner (Axio Scan Z.1, Zeiss). Unbiased stereological counting in the SNpc was performed on 8 brain sections per mouse (from C1 to C8) to obtain the entire structure, then the number of cells was approximated by integral calculation. Positive cells were counted using artificial intelligence (Aiforia).

### Antibodies

The following primary antibodies were used: rabbit anti-TH (Novus Biologicals, NB300-109) at 1:2500; mouse anti-HA (Biolegend, 901516) at 1:1000. Secondary antibodies for immunoblotting were the appropriate goat anti-mouse or anti-rabbit Alexa-Fluor 488 or Alexa-Fluor 647 (Invitrogen)

### Results:

First, the inventors demonstrated that administration of the AAV-CYP46A1 vector in the striatum allows, by retrograde transport, the expression of the transgenic CYP46A1 protein in the dopaminergic neurons (Tyrosine Hydroxylase (TH)-positive) of the SN (data not shown). Stereotaxic injection in mice is well tolerated. The expression of CYP46A1 in the striatum and the SNpc is rapid (plateau at D21 after injection) and stable (analyzed up to 12 months in mice and 6 months in primates). The enzyme is functional (increased 24OHC) and does not cause toxicity (data not shown).

Then, the inventors demonstrate a significant reduction of CYP46A1 expression in the SNpc of a PD mouse model, the MPTP model. MPTP is the only known dopaminergic neurotoxin capable of causing a clinical picture in humans and monkeys indistinguishable from PD. Its use is not technically difficult: it does not require any special equipment such as a stereotaxic frame, nor any surgical intervention on living animals such as 6-hydroxydopamine or rotenone. It is a reliable product and damage to the nigrostriatal dopaminergic pathway is reproducible, which is often not the case with other known toxins (Jackson-Lewis 2007).

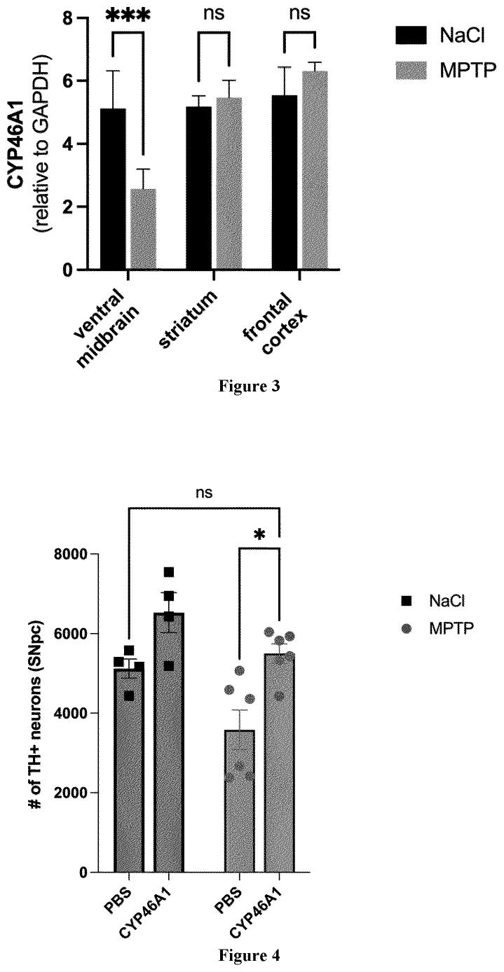

CYP46A1 expression is reduced in the SNpc of MPTP mice 3 days after the last MPTP injection and there was no reduction of CYP46A1 expression in the striatum or a control region (frontal cortex) 3 days after the last injection (FIG. 3).

The inventors also observed a protection of the TH-positive neurons loss induced by the CYP46A1 overexpression by immunofluorescence 30 days after the last MPTP injection in the whole SNpc (FIG. 4). Indeed, MPTP intoxicated mice have an average of 3500 TH+ neurons per SNpc versus an average of 5500 TH-positive neurons with AAV-CYP46A1 treatment (of note, a C57BL/6J mouse has an average of 6000-7000 TH+ neurons per SNpc). This protection seems to be more significant in the SNpc than in other dopaminergic regions of the midbrain (data not shown).

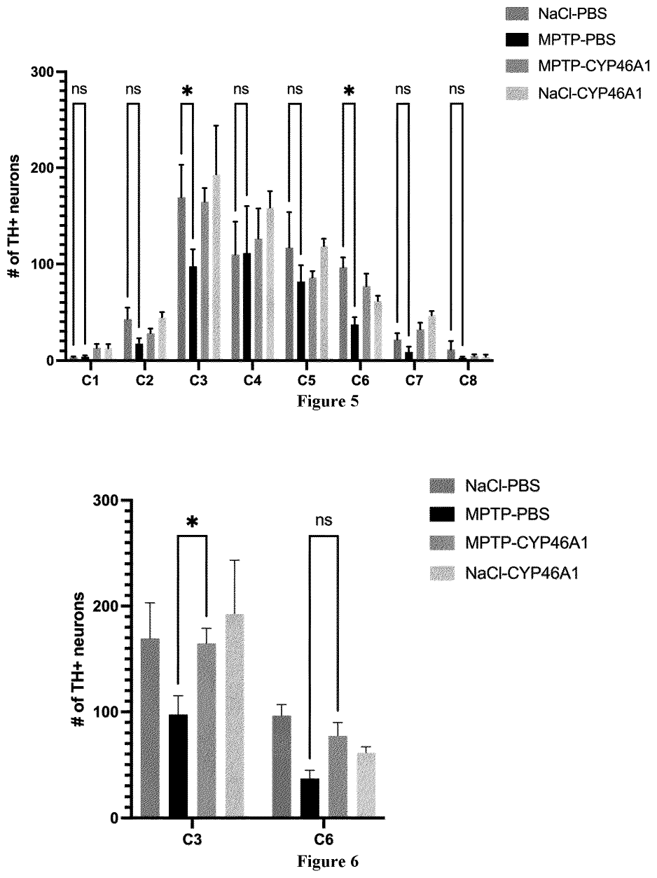

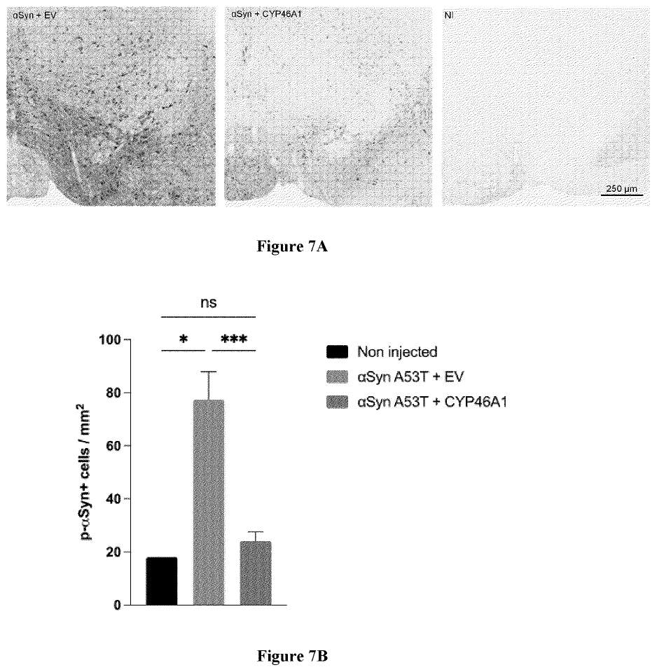

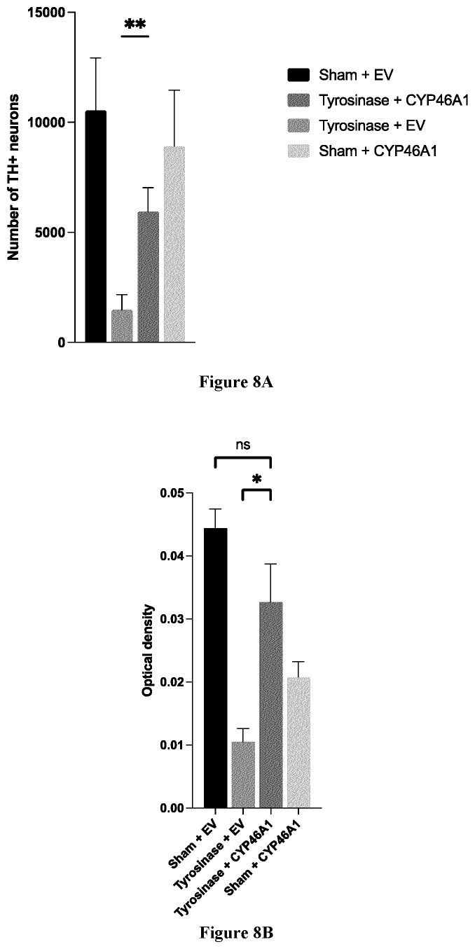

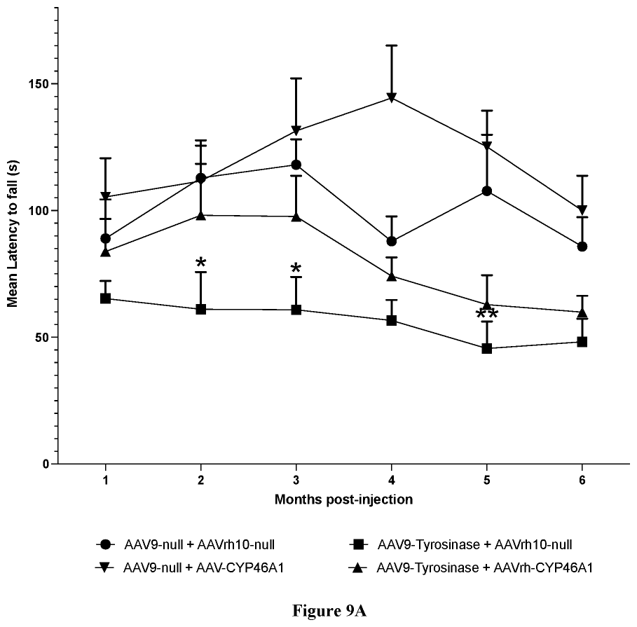

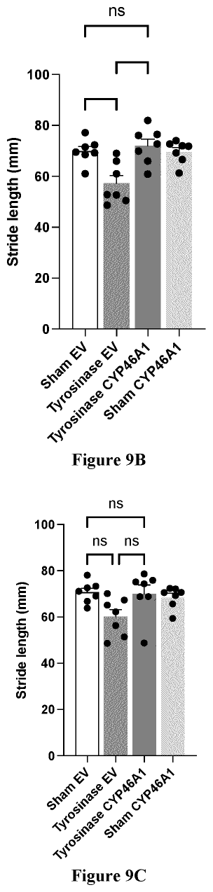

In addition, among the regions of the SNpc significantly affected by MPTP intoxication (C3 and C6, FIG. 5), a significant protection by the overexpression of CYP46A1 is observed by the counting of TH-positive neurons in ½ of the SNpc damaged by the MPTP and a trend of protection in the other ½ (FIG. 6).

### Example 4: Alpha-Synuclein Vector-Based Model

### Material and Methods

### Stereotaxic Injections of AAVs in the Mouse Brain

C57BL6/J 8-week-old mice were first analgesized by a subcutaneous injection of buprenorphine, then placed in an induction cage saturated with 4%-20% isoflurane-02 for 2 to 4 minutes and anesthesia maintained by inhalation of isoflurane in a face mask (1-1.5%-20%). Mice were positioned on a stereotaxic frame (Kopf) equipped with a 10 μL 1701 Hamilton syringe (Dutscher, 074493) and a tailor-made 32-gauge needle (Dutscher, 074753). A mixture of AAV9-SNCA-A53T and AAVrh. 10-CYP46A1 or AAVrh. 10-CAG-null recombinant vectors was injected bilaterally into the mouse SNpc in a final volume of 1 μL at a rate of 0.2 μL/minute. Viral preparation corresponding to 1.10⁹ vector genomes (vg)/SNpc for the AAVrh. 10-CYP46A1 or the AAVrh. 10-null and 1.5.10¹⁰ vg/SNpc for the AAV9-SNCA-A53T were injected into the right and the left SNpc at the stereotaxic coordinates: 2.9 mm caudal to the bregma, 1.3 mm lateral to midline and 4.2 mm ventral to the skull surface. The pipette was left in place for 5 min after injection to avoid leakage. After surgery, mice were placed in an incubator for optimized recovery from anaesthesia.

### AAV Plasmid Design and Vector Production

Vector production has been described elsewhere (Hudry et al., 2010). The viral constructs for AAVrh. 10-null and AAVrh. 10-CYP46A1-HA contained the expression cassette consisting of the human Cyp46a1 genes, driven by a CMV early enhancer/chicken β-actin (CAG) synthetic promoter (CAG) surrounded by inverted terminal repeats (ITR) sequences of AAV2. These vectors were produced and purified by Atlantic Gene therapies (INSERM U1089, Nantes, France).

AAV9-SNCA-A53T contained the expression cassette consisting of the human alpha-synuclein gene (SNCA) with the A53T mutation, driven by the human synapsin 1 (SYN1) synthetic promoter surrounded by inverted terminal repeats (ITR) sequences of AAV2, was produced and purchased on Vector Builder (P201129-1003).

### Tissue Collection

Mice were anesthetized with pentobarbital (Euthasol 180 mg/kg) solution and perfused transcardially with phosphate buffered saline (PBS). Brains were collected and post-fixed in PFA 4% prior to inclusion for histology analysis.

### Histological Analysis

Coronal brain sections (20 μm) were cut on a freezing microtome (Leica), collected serially in a cryoprotective solution, and stored at −20° C. until use. Immunostainings were carried out on frozen free-floating sections. Slices were washed in PBS 0.1M and blocked in PBS 0.1M Triton 0.03% containing 5% normal goat serum (NGS, Gibco) for 1 hour. Sections were then incubated with the respective primary antibody (see Antibodies section) in PBS-Triton 0.03% containing 3% NGS over night at 4° C. After three successive washes of 5 minutes in PBS, brain slices wete incubated for 1 hour and 30 minutes with secondary Alexa Fluor-conjugated (Invitrogen) antibodies directed against the species of the primary antibody at room temperature. Slices were stained in a DAPI solution, mounted in Fluoromount-G™ (Invitrogen) and conserved at 4° C. until microscopy analysis. The immunoassayed slides were then scanned using a NanoZoomer S60 digital slide scanner (Hamamatsu, C13210-01) at 20× magnification. Unbiased stereological counting in the SNpc was performed on 8 brain sections per mouse (from C1 to C8) to obtain the entire structure, then the number of cells was approximated by integral calculation. Positive cells were counted using artificial intelligence (Aiforia).

### Antibodies

The following primary antibody was used: rabbit anti-S129-alpha-synuclein (Abcam, ab51253). Secondary antibody for immunochemistry was the appropriate biotinylated goat anti-rabbit (Invitrogen).

### Results:

The inventors investigated in vivo the impact of CYP46A1 in a mouse model of synucleopathy. To this end, an AAV vector (AAV9-SNCA-A53T) encoding for a mutated form of alpha-synuclein that is more prone to aggregation was injected into the SNpc of mice. This vector was co-administered with a vector encoding CYP46A1 (AAV.rh10-CYP46A1) or a control vector not encoding any protein (“empty vector”, EV: AAV.rh10-null). The inventors wanted to demonstrate whether CYP46A1 could influence the onset and spread of alpha-syn pathology (aggregation of alpha-synuclein). To this end, the form of alpha-synuclein phosphorylated on serine 129 was detected by immunohistochemistry on mouse brain sections from the midbrain, including the SNpc. This phosphorylated form of alpha-synuclein (phospho-αSyn) is the in vivo marker of its aggregation. This immunorevelation was done in non-injected control mice (NI), alpha-synuclein non-treated mice (αSyn+EV) given AAV9-SNCA-A53T and AAVrh. 10-null vectors, and alpha-synuclein treated mice (αSyn+CYP46A1) given AAV9-SNCA-A53T and AAVrh. 10-CYP46A1 vectors. The inventors show a visible decrease in phospho-αSyn in mice treated with CYP46A1 (FIG. 7A, right panel).

The number of cells with positive phospho-αSyn inclusions was next assessed in the midbrain of alpha-synuclein mice treated or not with AAV-CYP46A1 and the inventors show a significant decrease in the number of phospho-αSyn positive cells in mice treated with CYP46A1 (FIG. 7B). This confirms the positive role of CYP46A1 in autophagy and in the proteostatic mechanisms that help to manage alpha-synuclein aggregates in neurons.

This suggests that CYP46A1 could have a beneficial effect in PD mice models, particularly in the synucleinopathy models.

### Example 5: Neuromelanin Vector-Based Model

### Stereotaxic Injections of AAVs in the Mouse Brain

C57BL6/J 8-week-old mice were first analgesized by a subcutaneous injection of buprenorphine, then placed in an induction cage saturated with 4%-20% isoflurane-02 for 2 to 4 minutes and anesthesia maintained by inhalation of isoflurane in a face mask (1-1.5%-20%). Mice were positioned on a stereotaxic frame (Kopf) equipped with a 10 μL 1701 Hamilton syringe (Dutscher, 074493) and a tailor-made 32-gauge needle (Dutscher, 074753). A mixture of AAV9-Tyrosinase and AAVrh. 10-CYP46A1 or AAVrh. 10-null recombinant vectors was injected bilaterally into the mouse SNpc in a final volume of 1 μL at a rate of 0.2 μL/minute. Viral preparation corresponding to 1.10⁹ vg/SNpc for the AAVrh. 10-CYP46A1 or the AAVrh. 10-null and 4.5.10⁹ vg/SNpc for the AAV9-Tyrosinase were injected into the right and the left SNpc at the stereotaxic coordinates: 2.9 mm caudal to the bregma, 1.3 mm lateral to midline and 4.2 mm ventral to the skull surface. The pipette was left in place for 5 min after injection to avoid leakage. After surgery, mice were placed in an incubator for optimized recovery from anaesthesia.

### AAV Plasmid Design and Vector Production

Vector production has been described elsewhere (Hudry et al., 2010). The viral constructs for AAVrh. 10-null and AAVrh. 10-CYP46A1-HA contained the expression cassette consisting of the human Cyp46a1 genes, driven by a CMV early enhancer/chicken β-actin (CAG) synthetic promoter (CAG) surrounded by inverted terminal repeats (ITR) sequences of AAV2. These vectors were produced and purified by Atlantic Gene therapies (INSERM U1089, Nantes, France).

AAV9-Tyrosinase contained the expression cassette consisting of the human Tyrosinase gene, driven by the CMV synthetic promoter surrounded by inverted terminal repeats (ITR) sequences of AAV2, or the AAV9-null, were kindly provided by Dr José Luis Lanciego and produced by the University of Barcelona (Spain).

### Mice Behavior Assessment

Rotarod: Mice were tested once a month from 1-month to 6-month post-injection. Each daily session included a 5-minute training trial at 4 rpm on the rotarod apparatus (Bioseb). 30 minutes later, mice were tested for three consecutive accelerating trials with the speed linearly increasing mode (from 4 to 40 rpm overs 300 seconds). The latency to fall from the rod (duration in seconds) was recorded for each trial.

Gait analysis: Mice were acclimated in the testing room for 30 minutes prior to recordings. Mice were placed in the corridor (Runway, CleverSys Inc.). Once the mouse entered in the view of the camera located below the walkway, it recorded for one passage at 2000 frames per second (AVI format). The recordings were then viewed and analysed blind using Fiji (ImageJ) software.

### Tissue Collection

Mice were anesthetized with pentobarbital (Euthasol 180 mg/kg) solution and perfused transcardially with phosphate buffered saline (PBS). Brains were collected and post-fixed in PFA 4% prior to inclusion for histology analysis.

### Histological Analysis

Coronal brain sections (20 μm for the SNpc, 30 μm for the striatum) were cut on a freezing microtome (Leica), collected serially in a cryoprotective solution, and stored at −20° C. until use. Immunostainings were carried out on frozen free-floating sections. Slices were washed in PBS 0,1M and blocked in PBS 0.1M Triton 0.03% containing 5% normal goat serum (NGS, Gibco) for 1 hour. Sections were then incubated with the respective primary antibody (see Antibodies section) in PBS-Triton 0.03% containing 3% NGS over night at 4° C. After three successive washes of 5 minutes in PBS, brain slices wete incubated for 1 hour and 30 minutes with seconday Alexa Fluor-conjugated (Invitrogen) antibodies directed against the species of the primary antibody at room temperature. Slices were stained in a DAPI solution, mounted in Fluoromount-G™ (Invitrogen) and conserved at 4° C. until microscopy analysis. The immunoassayed slides were then scanned using a slide scanner (Axio Scan Z.1, Zeiss). Unbiased stereological counting in the SNpc was performed on 8 brain sections per mouse (from C1 to C8) to obtain the entire structure, then the number of cells was approximated by integral calculation. Positive cells were counted using artificial intelligence (Aiforia). Optical density of dopaminergic terminals in the striatum was measured using Fiji software (ImageJ).

### Antibodies

The following primary antibodies were used: rabbit anti-TH (Novus Biologicals, NB300-109) at 1:2500; mouse anti-HA (Biolegend, 901516) at 1:1000. Secondary antibodies for immunoblotting were the appropriate goat anti-mouse or anti-rabbit Alexa-Fluor 488 or Alexa-Fluor 647 (Invitrogen)

### Results:

The inventors investigated in vivo the impact of CYP46A1 in a recent mouse model of Parkinson's disease, the Neuromelanin mouse model. The principle of this model lies in the fact that the Tyrosinase enzyme, which is responsible for the production of neuromelanin (NM), is expressed in the SNpc of mice using an AAV vector. In humans, dopaminergic neurons expressing NM are particularly susceptible to degeneration. NM is absent from the substantia nigra of rodents, and over-expressing it leads to a PD-like phenotype. Interestingly, it has been shown that p62-positive Lewy body-like inclusions are found in the substantia nigra of rodents in this model (Carballo-Carbajal et al., 2019).

To this end, an AAV vector (AAV9-Tyrosinase) or its control (Sham, AAV9-null) was injected into the SNpc of mice. This vector was co-administered with a vector encoding CYP46A1 (AAV.rh10-CYP46A1) or a control vector not encoding any protein (EV, AAV.rh10-null). First, the authors show that CYP46A1 significantly prevents the loss of dopaminergic neurons in the SNpc of Neuromelanin mice (FIG. 8A). Tyrosinase mice treated with AAVrh. 10-CYP46A1 lost significantly fewer neurons than Tyrosinase mice treated with AAVrh. 10 control. This significant reduction in the loss of dopaminergic neurons is correlated with the prevention of the loss of dopamine endings in the striatum (FIG. 8B). It should be noted that dopaminergic degeneration is particularly strong in this model, and yet is reduced by AAVrh. 10-CYP46A1.

The loss of around 50% of the dopaminergic neurons in the SNpc and around 50% of the dopaminergic endings in the striatum is necessary for motor symptoms to appear in humans (Kordower et al., 2013). In the Neuromelanin mouse model, the inventors show a reduction in motor function as assessed by Rotarod test (FIG. 9A) and in the analysis of spontaneous gait (FIGS. 9B-9C). Impairment of motor function assessed by rotarod in Tyrosinase mice was significant at 2-, 3- and 5-months post-injection of AAVs. At these time points, CYP46A1-treated Tyrosinase mice showed no significant impairment of rotarod performance, demonstrating that CYP46A1 prevents this impairment (FIG. 9A). In addition, the forepaw gait of Tyrosinase mice is significantly impaired, as can be seen through the increase in stride length. This gait impairment, more specific to the pathology, is significantly prevented by CYP46A1 treatment (FIG. 9B). The decrease in stride length is only significant for the front (not hind) limbs in Tyrosinase mice, although there is a trend for the hind legs, also prevented by CYP46A1. This is expected when lateral SNpc damage is induced, which is explained by the AAV-Tyrosinase vector injection coordinates used.

This suggests that CYP46A1 could have a beneficial effect on PD mouse models, in particular on models that reproduce cellular mechanisms close to those producing Lewy bodies in humans.

### Conclusion:

All these data identify for the first time CYP46A1 as a relevant therapeutic target for alpha-synucleinopathies and more particularly for Parkinson disease and the useful of a vector comprising the full sequence of cholesterol 24-hydroxylase encoding nucleic acid to treat these diseases.

### REFERENCES

Throughout this application, various references describe the state of the art to which this invention pertains. The disclosures of these references are hereby incorporated by reference into the present disclosure.
Burlot M A. Et al. Cholesterol 24-hydroxylase defect is implicated in memory impairments associated with Alzheimer-like Tau pathology. Hum Mol Genet 2015 Nov. 1; 24(21):5965-76.
Cassia N. Cearley. Transduction characteristics of adeno-associated virus vectors expressing cap serotypes 7, 8, 9, and Rh10 in the mouse brain. Molecular Therapy 2009. Jackson-Lewis Vernice, Serge Przedborski. Protocol for the MPTP mouse model of Parkinson's disease. Nat Protoc 2007.
Hudry E. et al. Adeno-associated virus gene therapy with cholesterol 24-hydroxylase reduces the amyloid pathology before or after the onset of amyloid plaques in mouse models of Alzheimer's disease. Mol Ther. 2010 January; 18(1):44-53. doi: 10.1038/mt.2009.175. Epub 2009 Aug. 4.
Wurtz G. et al. CYP46A1 as a Relavant Target to Treat ALS Pathology Independent from its Origin. Molecular Therapy 30 (4), 569-569, 2022.
Carballo-Carbajal, I. et al. Brain tyrosinase overexpression implicates age-dependent neuromelanin production in Parkinson's disease pathogenesis (2019). Nat Commun 10, 973.

1-8. (canceled)

9. A pharmaceutical composition formulated for use in the treatment of alpha-synucleinopathies in a subject in need thereof which comprises a therapeutically effective amount of a vector comprising a full sequence of cholesterol 24-hydroxylase encoding nucleic acid.

10. A method of treating alpha-synucleinopathies in a subject in need thereof, comprising administering to the subject a therapeutically effective amount of a vector which comprises a full sequence of cholesterol 24-hydroxylase encoding nucleic acid.

11. The method according to claim 10, wherein the vector comprises a nucleic acid sequence that encodes the amino acid sequence SEQ ID NO:2.

12. The method according to claim 10, wherein the vector comprises a nucleic acid sequence SEQ ID NO:1 or a variant thereof.

13. The method according to claim 10, wherein the vector is selected from the group of consisting of adenovirus, lentivirus, retrovirus, herpesvirus and Adeno-Associated Virus (AAV) vectors.

14. The method according to claim 10, wherein the vector is an AAV vector.

15. The method according to claim 14, wherein the AAV vector is an AAV 10 vector or an AAVrh.10 vector.

16. The method according to claim 10, wherein the vector is administered intravenously.

17. The method according to claim 10, wherein the alpha-synucleinopathie is Parkinson's disease (PD), Parkinson's disease with dementia, dementia with Lewy bodies or multiple-system atrophy (MSA).
